(19) United States
(12) Patent Application Publication
(10) Pub. No.: US 2007/0172489 A1
(43) Pub. Date: Jul. 26, 2007

(54) Caspase inhibitors, especially caspase 3 inhibitors, for the treatment of influenza

(76) Inventors: Stefan Ludwig, Wurzburg (DE); Oliver Planz, Rottenburg (DE); Hans-Harald Sedlacek, Marburg (DE); Stephan Pleschka, Giessen (DE)

Correspondence Address: MAYER & WILLIAMS PC, 251 NORTH AVENUE WEST, 2ND FLOOR, WESTFIELD, NJ, 07090, US

(21) Appl. No.: 10/550,856

(22) PCT Filed: Mar. 24, 2004
(86) PCT No.: PCT/DE04/00646

§ 371 Date: Jul. 10, 2006

(30) Foreign Application Priority Data
Mar. 26, 2003 (DE) 103 13 636.3

(51) Int. Cl. A61K 48/00; A61K 38/16; C12Q 1/70; A61K 39/42

(52) U.S. Cl. 424/159.1; 514/12; 514/44; 435/5

(57) ABSTRACT

The invention relates to the use of at least one caspase inhibitor, in particular a caspase-3 inhibitor, for preparing a pharmaceutical composition for the prophylaxis and/or therapy of a viral infection, in particular an infection with an RNA negative-strand virus, preferably an influenza infection, and to a test system for identifying suitable active substances.

### FIELD OF THE INVENTION

The invention relates to the use of at least one active substance for the prophylaxis and/or therapy of a viral disease, wherein at least one active substance inhibits at least one cellular component such that a virus multiplication is inhibited. The present invention further relates to the combination of at least one such active substance with at least one further different antivirally acting substance for the prophylaxis and/or therapy of at least one viral disease.

### BACKGROUND OF THE INVENTION AND PRIOR ART

Infections with RNA or DNA viruses are a substantial threat for the health of man and animal. Infections with influenza viruses still belong to the big epidemics of mankind and cause year for year a large number of fatalities. They are an immense cost factor for the economy, for instance by inability to work because of illness. Of substantial economic importance are also infections with the Borna disease virus (BDV), in particular attacking horses and sheep, which was however already isolated in man, too, and which was connected here with neurological diseases.

The problem of controlling in particular RNA viruses is the adaptability of the viruses caused by a high fault rate of the viral polymerases, thus the preparation of suitable vaccines as well as the design of antiviral substances being very difficult.

Furthermore it has been found that the application of antiviral substances immediately directed against the functions of the virus, initially at the beginning of the therapy have a fair antiviral effect, but will lead very quickly to the selection of resistant variants, because of mutation. An example is the anti-influenza drug amantadine and its derivatives, which is or are directed against a transmembrane protein of the virus. Within a short time after application, resistant variants of the virus are generated.

Other examples are the new therapeutic agents for influenza reactions inhibiting the influenza-viral surface protein neuraminidase. Hereto belongs for instance Relenza. In patients, Relenza-resistant variants have already been found (Gubareva et al., J Infect Dis 178, 1257-1262, 1998). The hopes placed on this therapeutic agent thus could not be fulfilled.

Due to their in most cases very small genomes and therefore limited coding capacity for replication-necessary functions, all viruses have to rely to a strong extent on functions of their host cells. By exerting influence on such cellular functions being necessary for the viral replication, it is possible to negatively affect the virus replication in the infected cell. Then, there is no possibility for the virus to replace the missing cellular function by adaptation, in particular by mutation, in order to escape the selection pressure. This could already be shown for the example of the influenza A virus with relatively unspecific inhibiting substances against cellular kinases and methyltransferases (Scholtissek and Müller, Arch Virol 119, 111-118, 1991).

It is known that cells have a multitude of signal transduction pathways, by means of which signals acting on the cell are transmitted to the cell nucleus. Thereby the cell is able to react to outside stimuli with cell proliferation, cell activation, differentiation or controlled cell death.

These signal transduction pathways have in common that they include at least one kinase, which activates by phosphorylation at least one protein thereafter transducing the signal.

Observing the cellular processes induced after virus infections, it can be found that a multitude of DNA and RNA viruses activate in the infected host cell preferably a defined signal transduction pathway, the so-called Raf/MEK/ERK kinase signal transduction pathway or the MEK/SEK/JNK signal transduction pathway.

Newer data show that the inhibition of the Ras-Raf-MEK-ERK signal transduction pathway by active substances, which selectively inhibit one or several of kinases involved in this signal transduction pathway, for instance the MEK and/or the SEK, the intracellular multiplication of intranuclearly replicating negative-strand RNA viruses, for instance of influenza A virus and the Borna disease virus (BDV) (PCT/DE 01/01292; PCT/DE 02/02810).

It is known that viruses can inhibit the apoptosis of the infected cell. This could for instance be detected for influenza viruses in vitro and in vivo (Fesq et al., 1994; Hinshaw et al., 1994; Mori et al., 1995; Takizawa et al.; 1993). It could not fully be clarified, which virus protein acts proapoptotically therein, possibly the apoptosis of the host cell is induced by the generation of interferon (Balachandran et al., 2000) or by proapoptotic virus proteins such as PB1-F2 (Chen et al., 2001).

It is not clear, which influence the virus-induced apoptosis has on the virus multiplication. There are assumptions that the release of proapoptotic virus proteins may lead lymphocytes into the apoptosis, and that thereby a reduced immune defense against the virus-infected cells and a promotion of the virus multiplication may result (van Campen et al., 1989; Tumpey et al., 2000). Other assumptions are that the apoptosis of the host cell increases by the phagocytosis intensified thereby the immune reaction against the virus (Watanabe et al., 2002). On the other hand, it is known that an overexpression of the antiapoptotically acting Bcl-2 inhibits the virus multiplication (Hinshaw et al., 1994; Olsen et al., 1996). In contrast thereto are findings that the inhibition of the virus induced apoptosis by a caspase inhibitor did not have any influence on the synthesis of virus proteins (Takizawa et al., 1999).

The apoptosis of a cell may be induced, besides by viruses, also by different other proapoptotic mechanisms and proteins. It is common to these different mechanisms and proteins that they activate a proteolytic cellular cascade series of cysteinyl proteases, so-called caspases. The initially activated caspases such as caspase-8 and caspase-9 activate therein the effector cascades such as the caspases-3 and 6. These in turn cleave a series of cellular substrates and cause thereby the apoptosis of the respective cell (surveys in Cohen, 1997; Thornberry and Lazebnik, 1998).

### TECHNICAL OBJECT OF THE INVENTION

It is the object of the invention to provide active substances for pharmaceutical compositions, which show improved antiviral effects, and a test system for identifying such active substances.

### FINDINGS THE INVENTION IS BASED ON

Surprisingly it has been found that i) for multiplication, influenza viruses need the cellular caspases, in particular caspase-3, and that in cells without caspase-3, the virus-genome ribonucleic protein complexes cannot diffuse through the pores of the nucleus membrane into the cytoplasm, but remain in the nucleus, ii) the inhibition of at least one cellular caspase, in particular the inhibition of the caspase-3 will lead to a distinct inhibition of the multiplication of negative-strand RNA viruses, in particular of influenza viruses, and iii) the combination of an inhibitor for a caspase, in particular caspase-3, with another antivirally effective substance, for instance with an inhibitor for a cellular kinase, has a synergistic effect on the inhibition of the virus multiplication.

The surprising effects of caspase inhibitors on the multiplication of viruses, in particular of negative-strand RNA viruses, for instance of influenza viruses, is made clear by that this inhibition of the virus multiplication by the inhibition of the caspase is not connected with an inhibition of the synthesis of early or late virus proteins (for instance NP or NS1 (early) still from matrix proteins (M1, late)) and was still observed, when the caspase inhibitor was added only 4 h after the infection.

### BASICS OF THE INVENTION AND EMBODIMENTS

For achieving the technical object, the invention therefore teaches the subject matters of the patent claims, in particular i) the use of at least one active substance, which reduces the amount or activity of a cellular caspase, in particular caspase-3, for the prophylaxis and/or therapy of a viral disease, in particular of a viral disease caused by negative-strand RNA viruses, for instance by an influenza virus, ii) the combination of at least one active substance, which reduces the amount or activity of a cellular caspase, in particular caspase-3, with another antiviral active substance and the use of said combination for the prophylaxis and/or therapy of a viral disease, in particular of a viral disease caused by negative-strand RNA viruses, for instance by an influenza virus, iii) a test system for finding an active substance according to the invention, wherein said test system comprises: 1) a cellular caspase, preferably caspase-3, which is brought into contact with a test substance, and it is measured, whether the protease activity of the caspase reduces by the test substance, 2) a cell, which is brought into contact with a test substance, and it is measured, whether the amount or activity of a cellular caspase, preferably caspase-3, is reduced by the test substance, 3) a cell, which is infected with a virus, preferably with a negative-strand RNA virus, for instance an influenza virus, and to which thereafter a test substance is added, which is capable to reduce the amount or the activity of the cellular caspase, in particular caspase-3, and it is measured, whether this test substance can inhibit the multiplication of the viruses in said cell.

To the active substances in the meaning of the present invention belong for instance: peptide and non-peptide inhibitors of the cellular caspase-3, such as Z-DEVD-FMK, Ac-DEVD-CHO, Ac-DMQD-CHO, Z-D(OMe)E(OMe)VD(OMe)-FMK, Z-D(OMe)QMD(OMe)-FMK (all above from Alexis Biochemicals), inhibitors of cellular caspases, which can activate caspase-3, such as peptide and non-peptide inhibitors of the caspase-9, such as Z-LE(OMe)HD(OMe)-FMK, Z-LEHD-FMK, Ac-LEHD-CHO (all from Alexis Biochemicals), peptide and non-peptide inhibitors of the caspase-8, such as Z-LE(OMe)TD(OMe)-FMK, Ac-ESMD-CHO, Ac-IETD-CHO, Z-IETD-FMK (all from Alexis Biochemicals), peptide and non-peptide inhibitors of the caspase-10, such as Ac-AEVD-CHO, Z-AEVD-FMK (both from Alexis Biochemicals), peptide and non-peptide inhibitors of other caspases or granzyme B and pan-caspase inhibitors, such as Z-VAD-FMK, Z-VAD-(OMe)-FMK (both from Alexis Biochemicals), Ac-YVAD-CHO, Z-YVAD-FMK, Z-VDVAD-FMK, Ac-LEVD-CHO (all from Calbiochem), dominant negative mutants of a cellular caspase, in particular of the caspase-3, antisense-oligonucleotides, which specifically accumulate at the DNA sequence or m-RNA sequence coding for a cellular caspase and inhibit the transcription or translation thereof, dsRNA oligonucleotides, which are suitable for the specific degradation of the mRNA's of a cellular caspase by the RNAi technology according to the methods as described by Tuschl et al. (Genes Dev 13:3191-3197, 1999) and Zamore et al. (Cell 101:25-33, 2000), antibodies or antibody fragments specific for a cellular caspase, or fusion proteins containing at least one antibody fragment, for instance a Fv fragment, which inhibit the protease activity of at least one caspase, inhibitors, which indirectly inhibit the expression or the activation of cellular caspases, in particular caspase-3, expression of proteins, which inhibitingly act on caspases, for instance the cellular inhibitors of apoptosis proteins cIAP1, cIAP2, the X-linked inhibitor of apoptosis protein XIAP, the antiapoptotic protein Bcl-2 or the baculoviral protein p35.

Preferably, the use of at least one active substance according to the invention occurs at the occasion of a viral disease, which is caused by RNA or DNA viruses, preferably negative-strand RNA viruses, for instance influenza viruses, or Borna viruses.

Another embodiment of the present invention relates to a combination preparation for the prophylaxis and/or therapy of at least one viral disease, containing at least two antiviral active substances, wherein at least one active substance inhibits a cellular caspase, preferably the caspase-3, and at least one further antiviral active substance.

To further antiviral active substances belong for instance 1-adamantanamine(amantadine), rimantadine, neuraminidase inhibitors such as Relenza, synthetic nucleoside analogs such as 3-deazaadenosine and ribavirin, antivirally acting inhibitors of the cellular kinases, for instance described in the patent applications PCT/DE 01/01292 and PCT/DE 02/02810.

The administration of the combination preparation may take place as a mixture of the active substances. The active substances may however also be administered separately at the same place, for instance i.v., or at separate places, simultaneously or at different times within a period, wherein the substance administered first is still effective, for instance within a period of three days.

Another embodiment of the present invention relates to a test system for identifying active substances, which inhibit at least one cellular caspase, in particular the caspase-3, such that the multiplication of viruses, in particular of negative-strand RNA viruses, for instance of influenza viruses, comprising a. at least one cell infectable with at least one virus and comprising either at least one caspase, in particular caspase-3, and at least one virus infecting the cells, or b. at least one cell infectable with at least one virus and comprising at least one caspase, in particular caspase-3.

Cells in the meaning of the present invention are cells from different organs and tissues, for instance cells of the blood and lymphatic vessels, cells, which cover the body cavities. Equally comprised are cell cultures, in particular such, which can be acquired from cell banks, such as ATCC, in particular permissive, eukaryotic cell cultures, for instance A549 (homo sapiens) B82, NIH, 3T3, L929, all from Mus musculus, BHK from Cricetus cricetus, CHO from Cricetulus griseus, MDCK from Canis famliliaris, vero, COS-1 and COS-7, all from Cercopithecus aethiops, and primary embryo fibroblasts from Gallus gallus (CEF cells).

For instance, in the test system according to the invention for identifying active substances, it is tested by addition of substances, preferably in concentrations of 0.001 μmole to 100 μmoles, and viruses in a particle number, which can infect the selected cell, whether a substance is capable to inhibit the virus multiplication, without damaging the cell.

Preferably, the virus used in the test system according to the invention is an RNA or DNA virus, preferably an influenza virus.

In a preferred embodiment, the cell of the test system according to the invention contains at least one overexpressed caspase, in particular caspase-3, in particular by introduction of one gene or several genes influencing the caspase. By this overexpression, substances are detected, which can strongly inhibit caspases as well as intracellularly reach a high concentration for the inhibition of the overexpressed caspases.

For control purposes, the expression for at least one caspase, preferably the caspase-3, in a cell of a test system according to the invention is inhibited, for instance a) by the introduction of an antisense DNA or an antisense RNA, or b) by the introduction of at least one gene coding for at least one dominant-negative mutant of at least one caspase.

Another embodiment of the present invention relates to a method for identifying at least one active substance according to the invention for the prophylaxis and/or therapy of viral diseases, which inhibits the multiplication of viruses during viral diseases, comprising the following steps: a. bringing at least one test system according to the invention into contact with at least one potential active substance, and b. determining the effects on the virus multiplication.

Bringing into contact in the meaning of the present invention may for instance occur by addition of the active substances into the culture medium of a cell culture or by local or systemic administration of the active substances into an organism.

Bringing into contact in the meaning of the present invention also comprises the prior art methods, which permit the introduction of substances into intact cells, for instance infection, transduction, transfection and/or transformation and other methods known to the man skilled in the art. These methods are in particular preferred, if the substance comprises viruses, naked nucleic acids, for instance antisense DNA and/or antisense RNA, viroids, virosomes and/or liposomes, and virosomes and liposomes are also suitable to bring further active substances into the cell, besides a nucleic acid molecule.

The determination of the effects on the virus multiplication occurs for instance by plaque assays or comparison of virus titer-infected or non-infected cells.

Another preferred embodiment of the present invention relates to a method for preparing a drug for the prophylaxis and/or therapy of at least one viral disease, which substantially inhibits or inhibit the multiplication of viruses, comprising the following steps: a. performing a test system according to the invention, and b. reacting the active substance(s) with at least one auxiliary and/or additional substance.

Preferably, the active substance according to the invention is processed for the local or systemic administration to an organism by using methods known to the man skilled in the art and auxiliary and/or additional substances to a drug.

Suitable auxiliary and/or additional substances, which serve for instance for stabilizing or preserving the drug or diagnostic agent, are generally known to the man skilled in the art (see e.g. Sucker H et al. (1991) Pharmazeutische Technologie, 2^{nd} edition, Georg Thieme Verlag, Stuttgart). Examples for such auxiliary and/or additional substances are physiological common salt solutions, Ringer's dextrose, dextrose, Ringer's lactate, demineralized water, stabilizers, antioxidants, complex-forming agents, antimicrobial compounds, proteinase inhibitors and/or inert gases.

The local administration may for instance be made on the skin, on the mucous membrane, into a body cavity, into an organ, into a joint or into the connective or supporting tissue, by nasal administration or by inhalation. The systemic administration preferably occurs into the blood circulation, into the peritoneal cavity or into the abdominal cavity.

The drug preparation comprising the active substance according to the invention depends on the type of active substance and the way of administration and may for instance be a solution, a suspension, an ointment, a powder, a spray, or another inhalation preparation. Preferably, nucleotide sequences are inserted by methods well known to the man skilled in the art into a viral vector or a plasmid and reacted with auxiliary substances for the cell transfection. To these auxiliary substances belong for instance cationic polymers or cationic lipids. Antisense oligonucleotides are derivatized by methods familiar to the man skilled in the art, in order to protect them from enzymatic degradation by DNAses or RNAses.

The active substance according to the invention may be present in the form of a salt, ester, amide or as a pre-stage, and preferably only such modifications of the active substance are used, which do not cause any excessive toxicity, irritations or allergic reactions of the patient.

The active substance is mixed under sterile conditions with a physiologically acceptable carrier substance and potential preservation agents, buffers or driving agents, depending on the application. Such carrier substances for the drug preparations are familiar to the man skilled in the art.

Preferably, the active substance according to the invention is administered in a one-time dose, in particular preferably in several doses, and the individual doses do not exceed the maximum tolerable dose (MTD) of the respective active substance for man. Preferably, a dose is selected, which is half the MTD.

According to the present invention, the administration may take place either locally or systemically, only on one day or daily over several days or at every second or third day over several weeks, until a therapeutic effect is visible.

In the following, the invention will be explained in more detail with reference to examples representing embodiments only.

### EXAMPLE 1

### Virus Multiplication in Wildtype and in Caspase-3-Deficient Cells

In order to analyze whether caspases, in particular caspase-3, play an important role in the influenza virus multiplication, the activity and expression of the protease(s) was inhibited in four different ways: a) by addition of a cell-permeable inhibitor (Z-DEVD-FMK), which preferably inhibits the caspase-3 activity, besides other caspases, b) by expression of an inhibitory protein of caspases, XIAP (X-linked inhibitor of apoptosis) (Devereaux et al., Nature, 388, 300-304, 1997), which inhibits caspase-3, among others, c) by stable transfection of a vector, which forms a siRNA against the mRNA of caspase-3, d) by investigation of a cell line (MCF-7), which is caspase-3-deficient (Janicke et al., J Biol Chem, 273, 9357-9360) and which was complemented by a transient transfection with procaspase-3.

With regard to a), the following was made. MDCK cells were infected with the influenza A virus strain Bratislava/79 (fowl plague virus, FPV) with a multiplicity of infection of 1 (M.O.I.=1) in absence and presence of increasing amounts of the caspase-inhibitor Z-DEVD-FMK (2, 4, 20, 40 μM, Alexis Biochemicals), which preferably inhibits caspase-3. The concentration of DMSO corresponding to the highest inhibitor amount (2%) served as a solvent control. As another control, the inactive inhibitor analog Z-FA-FMK was used in a concentration of 40 μM. After 24 h, the cell supernatants were investigated with conventional methods (plaque titration) with regard to the amount of newly formed viruses. In parallel thereto, the effect of the caspase-inhibitor was analyzed by the measure of the cleavage of the cellular caspase-substrate PARP (poly-ADP ribose polymerase), which is cleaved for instance by caspase-3 (Tewari et al., Cell, 81, 801-809, 1995), in the Western blot of cell lysates. The effects of the inhibitor on the expression of early or late viral proteins (NS1, NP, M1) were also investigated in the Western blot. In a variant of the experiment, the inhibitor DEVD-FMK was added in a concentration of 40 μM, and was washed away already after 2 h after the infection and replaced by fresh medium, or was added 4 h only after the infection. In another modification of the experiment, the broadband caspase-inhibitor Z-VAD-FMK was used for comparison reasons in analogous concentrations in A549, MDCK as well as in vero cells.

With regard to b), the following was made. MDCK cells were transfected with a vector plasmid or with plasmids, which express XIAP or procaspase-3. The transfection was performed with the transfection reagent Lipofectamine 2000 (Life Technologies) according to standard methods (Ludwig et al., J Biol Chem, 276, 10990-10998, 2001). The transfection efficiencies were approx. 60%. 24 h after the transfection, the infection with the influenza A virus strain fowl plague virus (FPV) occurred with a multiplicity of infection of 1 (M.O.I.=1). Another 24 h after the infection, the titers of the newly formed viruses in the cell culture supernatant were investigated in standard plaque assays for MDCK cells. The successful expression of the transiently expressed proteins was verified in the Western blot.

With regard to c), the following was made. The lung epithelial cell line A549 was transfected by using standard methods (Lipofectamine 2000 (Life Technologies); Ludwig et al., J Biol Chem, 276, 10990-10998, 2001) with the vector pSUPER, which leads to the generation of small interfering dsRNA fragments in the cell (siRNA) (Brummelkamp et al., Science, 296, 550-553, 2002). As inserts were used the following target sequences of the caspase-3 (gene bank association No. NM004346): TGACATCTCGGTCTGGTAC (nt 417-435), CTGGACTGTGGCATTGAGA (734-755) and TAC-CAGTGGAGGCCGACTT (795-813) (clones #113, #252 and #311). In another clone (#313) was identified an insertion. Thus this clone served as a negative control. The constructs were transfected together with the vector pCAGI-puro, in order to make the cells selectable with the antibiotic puromycin. 24 h after the transfection, the cells were washed and then incubated with medium, which contained 1 μg/ml puromycin. 24 h later, the cells were intensely washed with PBS, and new antibiotic-containing medium was added. This procedure was then repeated for 7 days in presence of 0.6 μg/ml puromycin. After 7 days, the different cells were investigated for the expression of the caspase-3. Also after 7 days, the infection of the different cell lines with influenza A virus strain fowl plague virus (FPV) occurred with a multiplicity of infection of 1 (M.O.I.=1). Another 24 h after the infection, the titers of the newly formed viruses in the cell culture supernatant were tested in standard plaque assays for MDCK cells.

With regard to d), the following was made. The caspase-3-deficient breast carcinoma cell line MCF-7 was transfected with a vector plasmid or a plasmid, which expresses procaspase-3. The transfection was performed with the transfection reagent Lipofectamine 2000 (Life Technologies) according to standard methods (Ludwig et al., J Biol Chem, 276, 10990-10998, 2001). The transfection efficiencies were approx. 50%. 24 h after the transfection, the infection with the influenza A virus strain fowl plague virus (FPV) occurred with a multiplicity of infection of 1 (M.O.I.=1). Another 24 h after the infection, the titers of the newly formed viruses in the cell culture supernatant were investigated in standard plaque assays for MDCK cells. The successful expression of procaspase-3 was verified in the Western blot.

The following results were obtained:

a) The caspase-3-inhibitor Z-DEVD-FMK led in dependence on the dose to the reduction of the influenza virus titers after 24 h up to approx. 60% inhibition at a concentration of 40 μM. This inhibition precisely correlated with the obtained caspase inhibition, measured by the cleavage of the caspase substrate PARP. This shows that the level of the activity of cellular caspases, in particular of caspase-3, directly correlates with the efficiency of the virus multiplication, and that caspase inhibitors can be used for the inhibition of influenza multiplication. The same efficiency of the inhibition of the virus multiplication was obtained with a pan-caspase inhibitor, Z-VAD-FMK in A549 cells as well as in MDCK and vero cells, whereas an inactive inhibitor analog Z-FA-FMK did not show any effect. In addition was observed that in spite of the inhibiting effect on the virus multiplication, Z-DEVD-FMK had no significant effect on the virus protein expression. This is a piece of evidence for that caspase activity is needed relatively lately in the virus replication. This is supported by the finding that the inhibitor continued effectively inhibiting the virus multiplication, if it is added only 4 h after the infection, i.e. at late times of the replication cycle. The presence of Z-DEVD-FMK in the first two hours after the infection had no significant effects if removed thereafter.

b) The expression of the caspase-inhibitory protein XIAP led to a reduction of the influenza virus titer of approx. 50% after 24 h. This reduction substantially correlated with the obtained inhibition of the caspase activity, measured by the reduced cleavage of the protein PARP, which also was approx. 40-50% of the efficiency of the original activity. On the other hand, an increased virus multiplication could be observed in the transfected cells by expression of the procaspase-3. This once again verifies that the level of the caspase-3 activity in cells directly correlates with the efficiency of the influenza virus replication.

c)Western blot experiments showed that a stable expression of different siRNA segments in A549 cells led to a more or less strong reduction of the protein levels of caspase-3 in the different cell lines, whereas the expression of a control siRNA segment did not show any effects. According to the degree of reduction of the protein amount, gradual effects on the influenza virus replication in the different lines were obtained, and the strongest inhibiting siRNA segment (#113) led to an approx. 10-fold reduction of the virus titers. The control siRNA segment (#313) correspondingly not had any effect on the virus multiplication. It should be noted here that the strong expression inhibition of caspase-3 led to an increased expression and activity of the caspase-7 in the cell line #113. This effect, which can be understood as a compensation reaction of the cell, could however not eliminate the defects of the virus multiplication.

d) The infection of wildtype or vector-transfected MCF-7 cells led to very few titers only of descendant viruses, and this indicates that the replication efficiency of influenza A viruses in these caspase-3-deficient cells is very small. If however procaspase-3 was introduced by transient transfection into these cells, a 30-fold increase of the titers of descendant viruses was observed, another piece of evidence of the importance of caspases, in particular caspase-3 for an efficient influenza virus multiplication.

In total, these results have as a consequence that the degree of expression and activity of caspases, in particular caspase-3 directly correlates with the efficiency of the influenza virus replication. Correspondingly, caspases, in particular caspase-3, are target points for an anti-influenza virus prophylaxis or therapy.

### EXAMPLE 2

### Mechanism of the Inhibition of the Virus Multiplication by a Caspase Inhibitor

Western blot analyses of cell lysates of caspase inhibitor-treated influenza virus-infected cells showed that in spite of efficient inhibition of the virus multiplication, there was no effect on the viral protein synthesis, and thus a late step of the replication cycle, when the viral protein synthesis is substantially accomplished, seems to be affected by caspase activity (also see results for a)). This is supported by findings, which show that caspase inhibitors still have an efficient inhibition of the virus multiplication, if they are added 4 h only after the infection, i.e. at a late time in the infection cycle, whereas the presence of the substances in the first two hours of the infection did hot have any effect if removed thereafter. An essential step late in the infection cycle of influenza viruses is the export of newly formed viral RNA in the form of ribonucleic protein complexes (RNP's) from the cell nucleus of the infected cell. Recently could be shown that the caspase activity in the cells leads to an expansion of the nucleus pores, which permits a free diffusion of large proteins or protein complexes between cell nucleus and cytoplasm (Falerio and Lazebnik, J. Cell Biol, 151, 951-959, 2000). In order to analyze, whether caspase activity regulatingly influences the export of viral proteins or RNP's from the cell nucleus, and whether this happens by free diffusion of the proteins, the following experimental batches were performed: a) wildtype A549 cells and A549 cells, which carry a caspase-3 siRNA, were infected and tested in immunofluorescence analyses for the localization of the RNP's, b) wildtype MDCK cells were treated and infected with the caspase-3 inhibitor Z-DEVD-FMK, and were also tested in immunofluorescence analyses for the localization of the RNP's, c) MDCK cells were transfected with a plasmid for the influenza A virus nucleoprotein (NP), and the localization of the NP was tested in immunofluorescence analyses after apoptosis induction with staurosporine in presence and absence of a caspase inhibitor, d) MDCK cells were transfected for the reconstitution of RNP complexes with plasmids, which code for the NP and the influenza polymerases PB2, PB1 and PA, and with a plasmid, which expresses an influenza virus-specific RNA matrix. The effects on the localization of the RNP complexes were investigated in presence and absence of caspase inhibitors in immunofluorescence analyses.

With regard to a), the following was made. A549 cells or A549 cell lines, which carried the siRNA segment #113, were infected with the influenza A virus strain fowl plague virus (FPV) with a multiplicity of infection of 3 (M.O.I.=3). 5 h after the infection, the cells were forwarded by conventional methods (Pleschka et al., Nat cell Biol, 3, 301-305, 2001) to the immunofluorescence analysis with a goat anti-NP antiserum (Robert Webster, Memphis, USA) and an anti-goat Texas red-IgG secondary antibody (Dianova). The cell nuclei were stained with DAPI. The visualization occurred by an inverse fluorescence microscope in a magnification of 40.

With regard to b), the following was made. MDCK cells were infected with the influenza A virus strain fowl plague virus (FPV) with a multiplicity of infection of 5 (M.O.I.=5) in presence of DMSO (2%), caspase-3 inhibitor Z-DEVD-FMK (40 μM, Alexis Biochemicals), the inactive inhibitor analog Z-FA-FMK (40 μM, Alexis Biochemicals) or the MEK inhibitor U0126 (50 μM, Taros Coustom Biochemicals). 5 h after the infection, the cells were forwarded by conventional methods (Pleschka et al., Nat cell Biol, 3, 301-305, 2001) to the immunofluorescence analysis with a goat anti-NP antiserum (Robert Webster, Memphis, USA) and an anti-goat Texas red-IgG secondary antibody (Dianova). The cell nuclei were stained with DAPI, the staining of the cytoskeleton was made with phalloidin-FITC. The visualization occurred by an inverse fluorescence microscope in a magnification of 40.

With regard to c), the following was made. MDCK cells were transfected with plasmids, which code for the influenza A virus proteins PB2, PB1, PA and NP, and with a plasmid, which forms an antisense RNA for the green fluorescent protein accompanied by influenza virus-specific promoter elements as a matrix for the polymerase complex. It is known that the expression of these plasmids leads to a reconstitution of the RNP complexes, which is shown by the expression of the reporter gene, here GFP (Pleschka et al., J Virol, 70, 4188-4192, 1996). The transfection was performed with the transfection reagent Lipofectamine 2000 (Life Technologies) according to standard methods (Ludwig et al., J Biol Chem, 276, 10990-10998, 2001). 16 hours after the transfection, the cells were treated for 5 h with DMSO, staurosporine (1 M) and DMSO, staurosporine (1 M) and Z-DEVD-FMK (40 μM) or staurosporine (1 M) and leptomycin B (2 ng/ml). Then the cells were forwarded by conventional methods (Pleschka et al., Nat cell Biol, 3, 301-305, 2001) to the immunofluorescence analysis with a goat anti-NP antiserum (Robert Webster, Memphis, USA) and an anti-goat Texas red-IgG secondary antibody (Dianova). The cell nuclei were stained with DAPI. The visualization occurred by an inverse fluorescence microscope in a magnification of 40.

With regard to d), the following was made. MDCK cells were transfected with a plasmid, which codes for the influenza A virus NP. The transfection was performed with the transfection reagent Lipofectamine 2000 (Life Technologies) according to standard methods (Ludwig et al., J Biol Chem, 276, 10990-10998, 2001). 16 hours after the transfection, the cells were treated for 5 h with DMSO, staurosporine (1 M) and DMSO, staurosporine (1 M) and Z-DEVD-FMK (40 μM) or staurosporine (1 M) and U0126 (40 μM). Then the cells were forwarded by conventional methods (Pleschka et al., Nat cell Biol, 3, 301-305, 2001) to the immunofluorescence analysis with a goat anti-NP antiserum (Robert Webster, Memphis, USA) and an anti-goat Texas red-IgG secondary antibody (Dianova). The cell nuclei were stained with DAPI. The visualization occurred by an inverse fluorescence microscope in a magnification of 40.

The following results were obtained.

a) The comparison of the localization of the RNP complexes (according to the detection with an antiserum against the viral nucleoprotein (NP), the main component of the RNP's), showed that the infected A549 cells, which have in dependence from siRNA a strongly reduced expression of caspase-3, the RNP's are held back for 5 hours after the infection in the cell nucleus, whereas in the wildtype A549 cells at the same time the RNP's already accumulate efficiently in the cytoplasm. This shows that the degree of the caspase-3 expression correlates with the migration efficiency of the RNP's out of the cell nucleus and indicates that this effect is caspase-3-mediated.

b) The comparison of the localization of the RNP complexes in infected MDCK cells, which were incubated with solvent or different inhibitors, showed that the migration of the RNP's out of the cell nucleus 5 h after the infection can efficiently be inhibited by the caspase inhibitor Z-DEVD as well as by the MEK inhibitor U0126, not however by the inactive caspase inhibitor analog Z-FA-FMK. This is another piece of evidence that caspases, in particular caspase-3, mediate the efficient export of RNP's out of the cell nucleus.

c)After transient expression in unstimulated cells, the influenza virus NP showed a nuclear localization. If however in these cells the caspase activity was induced by addition of the apoptosis inductor staurosporine, the nucleoprotein could be found distributed over the complete cell. This “bleeding” out of the cell nucleus could be prevented by addition of the caspase-3 inhibitor Z-DEVD, not however by an inhibitor of the active nucleus export, leptomycin B. This indicates that the caspase activity mediates the free diffusion of large proteins presumably by a proteolytic expansion of the nucleus pores and thus promotes the migration of the NP into the cytoplasm.

d) After transient expression of the viral proteins PB2, PB1, PA and NP in MDCK cells, which, starting from a plasmid, in addition express an RNA matrix with influenza virus-specific promoter regions, which accompany a GFP reporter gene, cells with a green staining were found in the culture dish, which indicates that in these cells intact RNP complexes were formed. GFP as well as the RNP complexes were in the unstimulated cells in the nucleus. If however in these cells the caspase activity was induced by addition of the apoptosis inductor staurosporine, the GFP as well as the RNP complexes could be found again distributed over the complete cell. Correspondingly, this “bleeding” out of the cell nucleus could be prevented by addition of the caspase-3 inhibitor Z-DEVD, not however by an inhibitor of the active nucleus export, U0126. This indicates that the caspase activity mediates the free diffusion of very large protein complexes presumably by a proteolytic expansion of the nucleus pores and thus promotes the migration of the RNP's into the cytoplasm. Further, it is interesting that the respective cells, verified by the inhibitability with Z-DEVD-FMK, have caspase activity, however otherwise no morphologic signs of apoptotic cells, such as membrane bleeding or condensed nuclei. This shows that initial events of the apoptosis induction, such as early caspase activity, are already sufficient for mediating the better nucleus export of the protein complexes. Full execution of the apoptotic program is not necessary or would even be counterproductive.

### EXAMPLE 3

### The Synergistic Effect of a Caspase Inhibitor and a Kinase Inhibitor in the Inhibition of the Virus Multiplication

It is known that the export of influenza virus RNP's is mediated at least in part by the active nucleus export (O'Neill et al., EMBO J, 17, 288-296, 1998), and can correspondingly be inhibited by inhibitors of the active nucleus export machinery such as leptomycin B. It is also known that the RNP export can be inhibited in the late phases of the replication by inhibition of the Raf/MEK/ERK kinase cascade, for instance by the MEK inhibitor U0126, and here this is interfering with an active export mechanism. Surprisingly, it has been found in conjunction with the invention that the nucleus export of influenza virus RNP's can alternatively also be inhibited by caspase inhibitors, and here this is mainly blocking of a passive process.

Now it was intended to find out, whether a) the caspase-activating signal pathway and the Raf/MEK/ERK cascade influence each other, and whether b) by the inhibition of the active export by U0126 and the simultaneous inhibition of the increased passive diffusion by caspase inhibitors, i.e. so to speak blocking of two alternative export mechanisms, a synergistic inhibition effect on the influenza virus replication can be obtained.

With regard to a), the following was made. A549 cells were infected with the influenza A virus strain fowl plague virus (FPV) with a multiplicity of infection of 1 (M.O.I.=1) in presence of DMSO (2%), the caspase-3 inhibitor Z-DEVD-FMK (40 μM, Alexis Biochemicals) or the MEK inhibitor U0126 (40 μM, Taros Coustom Biochemicals). 24 h after the infection, the cells were lysated, and the lysates were forwarded by conventional methods (Pleschka et al., Nat cell Biol, 3, 301-305, 2001) to an anti-PARP Western blot for the determination of the caspase activity as well as to an ERK immunocomplex kinase assay for the determination of the activity of the Raf/MEK/ERK signal pathway in the infected and treated cells.

With regard to b), the following was made. A549 cells and caspase-3-deficient MCF-7 cells were infected with the influenza A virus strain fowl plague virus (FPV) with a multiplicity of infection of 1 (M.O.I.=1) in presence of DMSO (2%), the caspase-3 inhibitor Z-DEVD-FMK (40 μM, Alexis Biochemicals) or the MEK inhibitor U0126 (40 μM, Taros Coustom Biochemicals). 9 h and 24 h after the infection, the titers of the newly formed viruses in the cell culture supernatant were tested in standard plaque assays for MDCK cells.

The following results were obtained.

a) The inhibition of caspase-3 in infected cells led to a reduced cleavage of the caspase substrate PARP, not however to a reduced activity of the Raf/MEK/ERK signal pathway, measured by the degree of virus-induced activity of ERK in the immunocomplex kinase assay, which was substantially identical in DMSO and Z-DEVD-FMK-treated cells. Further, the inhibition of MEK by U0126 in concentrations, which efficiently inhibited the virus-induced activity of ERK, did not lead to a modified cleavage of PARP. This indicates that the caspase-3-dependent cascade and the Raf/MEK/ERK signal pathway mediate independently from each other different processes, which alternatively promote the RNP export and thus make the virus multiplication more efficient.

b) If in A549 cells simultaneously caspases, in particular caspase-3, were inhibited by Z-DEVD-FMK and the Raf/MEK/ERK cascade, a synergistic inhibitory effect on the virus replication could be observed after 9 h as well as after 24 h. Thereby, the suboptimum inhibitory effect of less than one power of ten by isolatedly used agents could be increased up to the >1 log step by a combined administration. In caspase-3-deficient MCF-7 cells, the Z-DEVD-FMK did not have any effect, as expected, on the virus multiplication. However, the already small titers of descendant viruses from these cells were again reduced by using U0126. These findings showed that in fact the caspase cascade and the Raf/MEK/ERK signal pathway regulate two alternative processes for the efficient support of the virus multiplication, and that according to these findings the combined application of caspase inhibitors and MEK inhibitors are ideally suited to inhibit the multiplication influenza viruses.

1. A method for the prophylaxis or therapy of at least one viral disease, comprising administering a physiologically effective dose of a pharmaceutical composition comprising at least one active substance that inhibits a cellular caspase such that a virus multiplication is inhibited.

2. The method of claim 1, wherein the caspase is caspase-3.

3. The method of claim 1, wherein the active substance(s) is (are) selected from the following active substances:
peptide and non-peptide inhibitors of the cellular caspase-3, comprising
Z-DEVD-FMK
Ac-DEVD-CHO
Ac-DMQD-CHO
Z-D(OMe)E(OMe)VD(OMe)-FMK
Z-D(OMe)QMD(OMe)-FMK,
inhibitors of cellular caspases, which can activate caspase-3, comprising
peptide and non-peptide inhibitors of the caspase-9, comprising
Z-LE(OMe)HD(OMe)-FMK
Z-LEHD-FMK
Ac-LEHD-CHO,
peptide and non-peptide inhibitors of the caspase-8, comprising
Z-LE(OMe)TD(OMe)-FMK
Ac-ESMD-CHO
Ac-IETD-CHO (Alexis Biochemicals)
Z-IETD-FMK,
peptide and non-peptide inhibitors of the caspase-10, comprising
Ac-AEVD-CHO
Z-AEVD-FMK,
peptide and non-peptide inhibitors of other caspases or granzyme B and pan-caspase inhibitors, comprising
Z-VAD-FMK
Z-VAD-(OMe)-FMK
Ac-YVAD-CHO
Z-YVAD-FMK
Z-VDVAD-FMK
Ac-LEVD-CHO,
an inhibitory peptide, comprising Z-VAD-FMK or Z-DEVD-FMK,
a non-peptide inhibitor of caspases,
dominant-negative mutant of a caspase,
an antisense-oligonucleotide, which specifically accumulates at the DNA sequence or m-RNA sequence coding for a cellular caspase and inhibits the transcription or translation thereof,
a protein, which inhibitingly acts on caspases, comprising the cellular inhibitors of apoptosis proteins cIAP1, cIAP2, the X-linked inhibitor of apoptosis protein XIAP, the antiapoptotic protein Bcl-2 or the baculoviral protein p35,
dsRNA oligonucleotides, which are suitable for the specific degradation of the mRNA's of a cellular caspase by the RNAi technology,
an antibody or antibody fragment specific for a caspase or a fusion protein containing at least one antibody fragment, comprising a Fv fragment, which inhibits the protease activity of a caspase.

4. The method of claim 1, wherein the viral disease is caused by RNA or DNA viruses, comprising influenza viruses.

5. A combination preparation for the prophylaxis or therapy of at least one viral disease, comprising at least two antiviral active substances, wherein at least one antiviral active substance is selected from the active substances according to claim 3, wherein the combination preparation can be used in the form of a mixture or as individual components for using them simultaneously or at different times at identical or different places.

6. A combination preparation for the prophylaxis or therapy of a viral disease, comprising at least one active substance according to claim 1 and at least one antivirally acting substance, which is a kinase inhibitor.

7. A combination preparation for the prophylaxis or therapy of a viral disease, comprising at least one active substance according to claim 1 and at least one antivirally acting substance, which is a 1-adamantanamine, a rimantadine, a neuraminidase inhibitor or a nucleoside analog comprising ribavirin.

8. The combination preparation according to claim 5 for the prophylaxis or therapy of an infection with negative-strand RNA viruses, comprising influenza viruses or Borna viruses.

9. A test system for finding active substances, which act on at least one cellular caspase, comprising caspase-3, such that a virus multiplication is inhibited, comprising:
a) at least one cell injectable with at least one virus and comprising at least one cellular caspase and at least one virus infecting the cells, or
b) at least one cell injectable with at least one virus and comprising at least one cellular caspase.

10. The test system according to claim 9, wherein the virus is an RNA or DNA virus, comprising an influenza virus.

11. The test system according to claim 9 wherein the cell comprises at least one overexpressed caspase, comprising caspase-3.

12. The test system according to claim 9, comprises a cell, in which at least one gene coding for at least one dominant-negative mutant of at least one caspase is expressed.

13. A test system according to claim 9, further comprising a cell in which the expression for at least one caspase, comprising caspase-3, is inhibited.

14. A method for identifying at least one active substance for the prophylaxis or therapy of viral diseases, which substantially inhibits or inhibit the multiplication of viruses during viral diseases, comprising the following steps:
a) bringing at least one test system according to claim 9 into contact with at least one potential active substance, and
b) determining the effects on virus multiplication.

15. A method for preparing a drug for the prophylaxis or therapy of a viral disease, which substantially inhibits the multiplication of viruses during viral diseases, comprising the following steps:
a) performing a test system according to claim 9, and
b) reacting the active substance(s) with at least one auxiliary and/or additional substance.

16. A method for the prophylaxis or therapy of a viral infection, comprising an infection with an RNA negative-strand virus, comprising an influenza infection, comprising administering a physiologically effective dose of a pharmaceutical composition, comprising at least one caspase inhibitor, comprising a caspase-3 inhibitor.

17. The method according to claim 16, wherein the pharmaceutical composition further comprises at least one additional antiviral active substance, which is not a caspase inhibitor, comprising an inhibitor of one or several cellular kinases.

18. A combination preparation for the treatment of a viral infection, comprising at least one caspase inhibitor and another antiviral active substance, which is not a caspase inhibitor, comprising an inhibitor of one or several cellular kinases, each in a physiologically well tolerated dose, and galenic auxiliary and carrier substances, wherein the caspase inhibitor and the further antiviral active substance exist in a mixture or in separate galenic preparations, intended for simultaneous or successive administration.

19. The combination preparation according to claim 18, wherein the caspase inhibitor is selected from the group consisting of the substances according to claim 3 and mixtures of such substances.

20. The use or a combination preparation according to claim 18, wherein the further antiviral active substance is selected from the group consisting of neuraminidase inhibitors, nucleoside analogs, 1-adamantanamine, rimantadine, ribavirin, Relenza, 3-deazaadenosine, MEK inhibitors, comprising butadiene derivatives, flavon derivatives and benzamide derivatives, 2-(2-amino-3-methoxyphenyl)-4-oxo-4H-(1)benzopyran, U0126, PD18453, PD98059, inhibitors of a kinase of the NF-kB signal transduction pathway, comprising non-steroidal anti-inflammatory substances inhibiting the NF-kB activity, comprising phenyl alkyl acid derivatives comprising sulindac or derivatives of sulindac comprising sulindac sulphoxide, sulindac sulphone, sulindac sulphide, benzylamide sulindac analogs, salicylic acid derivatives, comprising salicylic acid or acetysalicyl acid, salicylamide, salacetamide, ethenzamide, diflunisal, olsalazine or salazosulphapyridine, curcumin, antioxidants comprising pyrrolidine dithiocarbamate (PDTC), oxicams, comprising piroxicam, vitamin E and derivatives thereof, comprising pentamethyl hydroxychromane (PMC), 17 beta-oestradiol and derivatives thereof, polyphenoles of the tea comprising Epigallocatechin-3-gallate (EGCG), Bay11-7182, peptides, which inhibit the interaction of at least two components of the NF-kB signal transduction pathway, comprising peptides binding to NEMO, proteosome inhibitors, comprising PS-341 and lactacystin, antisense-oligonucleotides, which specifically accumulate at the DNA sequence or m-RNA sequence coding for a component of the NF-kB signal transduction pathway and inhibit the transcription or translation thereof, comprising antisense-nucleotide sequences specific for p65 or p50, dominant-negative mutants of a component of the NF-kB signal transduction pathway, dsoligonucleotides, which are suitable for the specific degradation of the mRNA's of a component of the NF-kB signal transduction pathway by the RNAi technology, antibodies and antibody fragments specific for a component of the NF-kB signal transduction pathway, or fusion proteins containing at least one antibody fragment, comprising a Fv fragment, which inhibit at least one component of the NF-kB signal transduction pathway, kinase-inhibiting flavon derivative or benzpyran derivative; kinase-inhibiting derivative of the 4H-1-benzopyran; flavopiridol derivative; 2-(2-amino-3-methoxyphenyl)-4-oxo-4H-(1)benzopyran; 7,12-dihydro-indolo (3,2-d)(1)benzazepin-6(5H)-on; 70H-staurosporine or a phosphokinase-inhibiting derivative of the 70H-staurosporine; butyrolactone; roscovitine; purvalanol A; emodin; anilinoquinazoline; phenylaminopyrimidine; trioylimidazole; paullone; [4-(4-fluorophenyl)-2-(4-methylsulfinylphenyl)-5-(4-pyridyl)1H-imidazole; [1,4-diamino-2,3-dicyano-1,4-bis(2aminophenylthio) butadiene; kinase-inhibiting derivative of the butadiene; [2-2′-amino-3′-methoxyphenyl)-oxa-naphtalen-4-on); [2-(2-chloro-4-iodo-phenylamino)-N-cyclo-propylmethoxy-3,4-difluoro benzamide; CEP-1347 (KT7515) bis-ethylthiomethyl; tetrapyrrolic macrocycles; pyrimidone derivative; 3-aminomethylen-indoline derivative; pyrazolo (3,4-b) pyridine derivative; pyrazole derivative; 1,4-substituted piperidirie derivative; lipoidic ammonium salt; dominant-negative mutant of a kinase of a cellular signal transduction pathway; antisense-oligonucleotide, which specifically accumulates at the DNA sequence or mRNA sequence coding for a kinase of a cellular signal transduction pathway and inhibits the transcription or translation thereof; dsoligonucleotides, which are suitable for degradation of the mRNA's from kinases of a cellular signal transduction pathway by the RNAi technology; antibodies and antibody fragments specific for a kinase or a fusion protein containing at least one antibody fragment, comprising a Fv fragment, which inhibits the kinase activity of a kinase module; or a peptide, which inhibits the interaction of at least two kinases activatable immediately after one another of a cellular signal transduction pathway, and mixtures of such substances.

21. A method for screening for prospective antiviral active substances, comprising the following steps:
a) a cell containing a caspase, comprising caspase-3, is infected with a virus, comprising an RNA negative-strand virus, comprising an influenza virus,
b) the cell is contacted with one or several prospective active substances,
c) viral replication in the cell is determined,
d) an active substance or a mixture of active substances is selected, if the viral replication measured in step c) is smaller than when executing steps a) to c) without a prospective active substance or with an inactive active substance,
e) a selected active substance is contacted with a cell infected with a virus, which does not express or contain a caspase, in particular caspase-3, and the viral replication is measured, and the active substance is further selected, if the measurement of the viral replication does not result in a significant modification relative to a measurement when contacting said infected cell with an inactive active substance or without any active substance,
wherein the steps a) and b) may occur in any order or simultaneously, and wherein the steps a) to d) on the one hand and the step e) on the other hand may occur in any order or simultaneously.
